(19) United States
(12) Patent Application Publication
(10) Pub. No.: US 2026/0366974 A1
(43) Pub. Date: Nov. 19, 2026

(54) MOISTURE METER AND METHOD FOR CONTROLLING MOISTURE METER

(71) Applicant: A&D Company, Limited, Tokyo (JP)

(72) Inventors: Shunsuke KASAMATSU, Saitama (JP); Kazuna OKABE, Saitama (JP); Kazuma FUNAHASHI, Saitama (JP)

(73) Assignee: A&D Company, Limited, Tokyo (JP)

(21) Appl. No.: 19/728,239

(22) Filed: Jul. 1, 2026

(30) Foreign Application Priority Data
Apr. 7, 2025 (JP) 2025-113467

(51) Int. Cl. G01N 5/04; G01N 1/44; G01N 33/44

(52) U.S. Cl. CPC G01N 5/045; G01N 1/44; G01N 33/442

(57) ABSTRACT

Provided is a moisture meter including a mass sensor, a heater, and an electronic circuit. The electronic circuit is configured to execute a heating temperature determination mode of heating a preliminary test sample for a predetermined time at each temperature stage while raising a heating temperature stepwise, recording a state of the preliminary test sample at each temperature stage, and, when a change in the state of the preliminary test sample occurs, determining a temperature at an immediately preceding temperature stage as a set heating temperature for a measurement sample. The preliminary test sample is the same substance as the measurement sample.

### TECHNICAL FIELD

The present disclosure relates to moisture meters and, more particularly, to a heat-drying moisture meter and a method for controlling a moisture meter.

### BACKGROUND ART

A heat-drying moisture meter has been known as a device for measuring the moisture content of a sample. The heat-drying moisture meter is a device that heats a sample to evaporate moisture in the sample, determines that the sample is sufficiently dried and stops heating when the mass change (decrease) of the sample becomes constant. The heat-drying moisture meter calculates the moisture content of the sample from the change in mass of the sample before and after heating and drying. Moisture content M [%] is obtained by equation (1).

M[%]=(W₀−D)/W₀*100 (1)

(where W₀ is the mass of the sample before heating and drying, and D is the mass of the sample after heating and drying.)

Some samples may undergo state changes upon heating, such as melting or discoloring including scorching. These state changes hinder the accurate measurement of moisture content. Therefore, it is important to appropriately set heating conditions, particularly a heating temperature.

Patent Literature 1 discloses a heat-drying moisture meter configured to set appropriate heating temperature of sample by raising the heating temperature stepwise, detecting a change in moisture content upon heating a preliminary test sample, calculating a parameter of a time function indicating the change in moisture content based on the detected change in moisture content, and determining that drying has ended when the parameter has changed.

### CITATION LIST

### Patent Literature

Patent Literature 1 JP 2002-286609 A1
Patent Literature 2 JP 2023-133689 A1

### SUMMARY

### Technical Problem

However, the moisture meter of Patent Literature 1 has a problem that a change in the parameter cannot be accurately evaluated, when a sample has a low moisture content, such as resin pellets, since a change in the moisture content due to heating is small.

The present disclosure has been made in view of such circumstances and has an object to provide a heat-drying moisture meter that can easily set an appropriate heating temperature for a sample having a low moisture content.

### Solution to Problem

To achieve the above object, a moisture meter according to one aspect of the present disclosure has the following configuration.
1. A moisture meter comprising: a mass sensor configured to measure a mass of a sample placed on a sample pan; a heater configured to heat the sample; and an electronic circuit configured to control the heater to heat the sample at a set temperature and calculate moisture content of the sample from a difference in mass of the sample before and after heating, wherein the electronic circuit is configured to execute a heating temperature determination mode of: heating a preliminary test sample for a predetermined time at each temperature stage while raising a heating temperature stepwise; recording a state of the preliminary test sample at each temperature stage; and when a change in the state of the preliminary test sample occurs, determining a temperature at an immediately preceding temperature stage as a set heating temperature for a measurement sample, and wherein the preliminary test sample is the same substance as the measurement sample.
2. In the aspect 1 described above, it is also preferable that the moisture further comprises: an openable/closable heater cover configured to cover the sample pan to form a heating chamber; an output interface configured to output information to a user; and an input interface configured to receive information from a user, wherein the electronic circuit instructs a user to open the heater cover to observe the state of the preliminary test sample at an end of each temperature stage via the output interface and to input the state via the input interface, to record the state.
3. In the aspects 1 and 2 described above, the measurement sample is preferably resin pellets.
4. In the aspects 1 to 3 described above, the electronic circuit is configured to update a heating condition with smaller temperature intervals between a temperature stage at which the change in the state of the preliminary test sample occurs and the immediately preceding temperature stage and to execute the heating temperature determination mode again using an updated heating condition.
5. In the aspects 1 to 4 described above, it is also preferable that the electronic circuit is configured to: set a target value of the moisture content of the measurement sample; heat the preliminary test sample at the set heating temperature for a predetermined time while monitoring the moisture content of the preliminary test sample; and set a time by which the moisture content reaches the target value as a measurement heating time.
6. In the aspects 1 to 5 described above, it is also preferable that the moisture meter further includes a camera configured to capture images of the preliminary test sample being measured at predetermined intervals, and the electronic circuit is configured to examine a state of the preliminary test sample based on the images and determine the temperature at the immediately preceding temperature stage immediately preceding to the temperature stage at which the change in the state of the preliminary test sample occurs as the set heating temperature for the measurement sample.

A control method for a moisture meter according to another aspect of the present disclosure has the following configuration.
7. A method for controlling a moisture meter, executed by a computer serving as an arithmetic control unit of the moisture meter that heats a sample at a set temperature and calculates a moisture content of the sample from a difference in mass of the sample before and after the heating, the method comprising: heating a preliminary test sample for a predetermined time at each temperature stage while raising a heating temperature stepwise; recording a state of the preliminary test sample at each temperature stage; and when a change in the state of the preliminary test sample occurs, determining a temperature at an immediately preceding temperature stage as a heating temperature for a measurement sample, wherein the preliminary test sample is the same substance as the measurement sample.

A storage medium according to another aspect of the present disclosure has the following configuration.
8. A non-transitory computer-readable storage medium including a program for causing a computer serving as an arithmetic control unit of the moisture meter to execute the method defined in the aspect 7.

### Advantageous Effects

According to the above aspects, it is possible to provide a heat-drying moisture meter that can easily set an appropriate heating temperature for a sample having a low moisture content.

### BRIEF DESCRIPTION OF DRAWINGS

FIG. 1 is a block diagram illustrating the configuration of a moisture meter according to a first embodiment.

FIG. 2 is a perspective view of the moisture meter, illustrating a state in which a heater cover is open.

FIG. 3 is a flowchart of an example of processing of the moisture meter in a heating temperature determination mode.

FIG. 4 is a diagram illustrating an example of a message displayed in the heating temperature determination mode, instructing recording of the state of a sample.

FIG. 5 illustrates an example of the measurement result screen displayed in the heating temperature determination mode.

FIGS. 6A-6D are photographs illustrating states of a preliminary test sample during heating in the heating temperature determination mode.

FIG. 7 illustrates an example of the heating temperature determination screen displayed in the heating temperature determination mode.

FIG. 8 is a flowchart for explaining a modification of processing in the heating temperature determination mode.

FIG. 9 is a configuration block diagram of a moisture meter according to another modification of the moisture meter.

FIG. 10 is a flowchart illustrating processing in the heating temperature determination mode in the moisture meter.

FIG. 11 is a block diagram illustrating the configuration of a moisture meter according to a second embodiment.

FIG. 12 is a flowchart illustrating an example of processing in a heating end time determination mode in the moisture meter.

FIG. 13A shows a moisture content-time profile during the heating end time determination mode, and FIG. 13B shows a plurality of moisture content-time profiles during measurement using the determined measurement heating times.

### DESCRIPTION OF EMBODIMENTS

Hereinafter, preferred embodiments of the present disclosure will be described with reference to the drawings. However, the present disclosure is not limited thereto. Hereinafter, in this description, a moisture meter means a heat-drying moisture meter unless otherwise specified. In addition, in the following description of the embodiment, members having the same mechanical configuration are denoted by the same reference numerals, and redundant description is appropriately omitted

### 1. First Embodiment

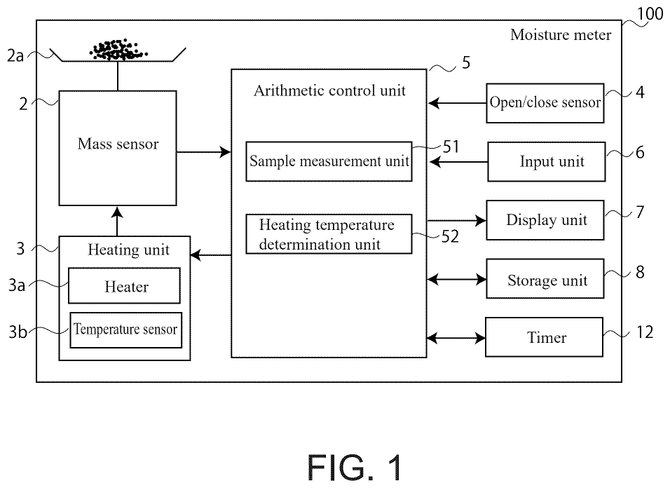

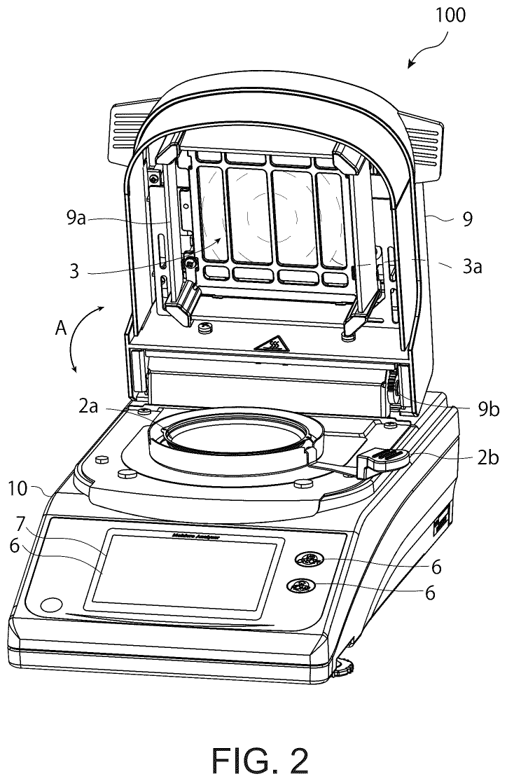

FIG. 1 is a block diagram illustrating the configuration of a moisture meter 100 according to the first embodiment. FIG. 2 is a perspective view of the moisture meter 100 illustrating a state in which a cover 9 is open. As illustrated in FIG. 1, the moisture meter 100 includes a sample pan 2a, a mass sensor 2, a heating unit 3, an open/close sensor 4, an arithmetic control unit 5, input units 6, a display unit 7, a storage unit 8, and a timer 12.

As illustrated in FIG. 2, the moisture meter 100 comprises a housing 10, the sample pan 2a, and the cover 9 in appearance. The housing 10 accommodates the mass sensor 2, the arithmetic control unit 5, and the storage unit 8 therein. The sample pan 2a is placed on the upper surface of the housing 10. The cover 9 has a container shape and configured to cover the sample pan 2a. The cover 9 is pivotably supported by a rotation shaft disposed behind sample pan 2a to open and close. The cover 9 can be opened and closed by rotated by a rotation shaft 9b disposed behind the sample pan 2a.

The cover 9 includes a rectangular container-shaped heater cover 9a, which is slightly smaller than the cover 9 therein. The heater cover 9a covers the sample pan 2a. Accordingly, the heater cover 9a is openable and closable integrally with the cover 9. The heating unit 3 is disposed between the heater cover 9a and the cover 9. The heater cover 9a is made of a transparent resin plate, for example. The heater cover 9a defines a heating chamber with the upper surface of the housing 10 in a closed state.

The mass sensor 2 may be implemented using an electromagnetic balance type sensor, a strain gauge type (load cell type) sensor, or a capacitance type electronic sensor. The mass sensor 2 is accommodated in the housing 10. The mass sensor 2 is connected to the sample pan 2a via a coupling shaft (not illustrated). The mass sensor 2 detects the mass of samples placed on the sample pan 2a. The mass sensor 2 is also connected to the arithmetic control unit 5 via an Analog/Digital (A/D) converter (not illustrated). The mass sensor 2 constantly outputs a mass detection signal to the arithmetic control unit 5.

The sample pan 2a is disposed inside the heating chamber. The sample pan 2a has a handle 2b and is configured to be attachable to and detachable from the mass sensor 2. The cover 9 is provided with the open/close sensor 4 that detects the opening/closing of the heater cover 9a. The open/close sensor 4 may be implemented using a micro switch such as a limit switch. The open/close sensor 4 is connected to the arithmetic control unit and outputs a detection signal to the arithmetic control unit 5.

The heating unit 3 comprises a heater 3a and a temperature sensor 3b. The heater 3a is implemented with a heater such as a halogen lamp or a resistance wire that generates Joule heat. The heater 3a is controlled by the arithmetic control unit 5 based on an output from the temperature sensor 3b. The heater 3a heats the heating chamber, i.e. samples in the heating chamber at a set temperature.

The input unit 6 is an input interface, such as an input switch, an input key, or, a physical input element. The input unit 6 may be configured integrally with the display unit 7, for example, as a touch panel display. The input unit 6 receives instructions and information from user to the arithmetic control unit 5. The input unit 6 may further includes a power button and a measurement stop button, etc.

The display unit 7 is an output interface. The display unit 7 may be implemented using a liquid crystal display, for example. The display unit 7 displays screens for outputting information such as measurement results to the user and for receiving instructions from the user.

The arithmetic control unit 5 is implemented using at least one electronic circuit, for example, a microcomputer comprising a central processing unit (CPU) that performs arithmetic processing, and a read only memory (ROM) and a random-access memory (RAM) mounted on an integrated circuit.

The arithmetic control unit 5 comprises a sample measurement unit 51 and a heating temperature determination unit 52 as functional units. Each functional unit may be configured by an electronic circuit such as a CPU, an application specific integrated circuit (ASIC), or a programmable logic device (PLD) such as a field programmable gate array (FPGA). In addition, when a functional unit is configured by a CPU, the function of the functional unit is implemented by reading a program for executing the function of the functional unit from the storage unit and executing the program.

The sample measurement unit 51 executes a sample measurement mode, which is used in normal operation, for measuring the moisture content of a sample. Specifically, the sample measurement unit 51 controls the heating unit 3 to heat the sample at a set heating temperature. During heating, the sample measurement unit 51 monitors the mass of the sample and a change in the mass of the sample. The sample measurement unit 51 stops the heating unit 3 to calculate the moisture content of the sample when the mass change rate of the sample becomes equal to or less than a predetermined threshold. The sample measurement unit 51 is also configured to stop the heating unit 3 to calculate the moisture content of the sample, when receiving an end signal from the timer 12, indicating that the measurement heating time set in advance has elapsed. Generally, when measuring samples having a low moisture content, such as resin pellets, the sample measurement unit 51 terminates measurement based on the timer 12.

In addition, the sample measurement unit 51 calculates the moisture content M of the measurement sample according to equation (2) given below using the mass measurement values of the sample before and after heating.

M(%)=[[(W1−W2)]/W1]×100 (2)

(where W1 represents the mass measurement value before heating and W2 represents the mass measurement value after heating.)

The heating temperature determination unit 52 executes a heating temperature determination mode for determining a heating temperature used when heating the measurement sample in the sample measurement mode. Specifically, the heating temperature determination unit 52 controls the heating unit 3 to heat a preliminary test sample, which is the same substance as the measurement sample. The heating temperature determination unit 52 raises the heating temperature stepwise and heats the preliminary test sample for a predetermined period of time at each temperature stage. The heating temperature determination unit 52 monitors the moisture content during heating. In addition, the heating temperature determination unit 52 may also monitor the moisture-content change rate (%/min) obtained by differentiating the moisture content with respect to time. The monitoring of the moisture-content change rate is not essential in this mode but is performed to grasp the dry state of the sample.

Table 1 is a table illustrating an example of heating conditions in the heating temperature determination mode. Under the conditions in Table 1, heating is performed for 5 minutes at each temperature stage while raising the heating temperature stepwise at 20 °C intervals, for example, at a heating temperature of 100 °C for 0 min to 5 min from the start of heating, at a heating temperature of 120 °C for 5 min to 10 min, and at a heating temperature of 140 °C for 10 min to 15 min. The heating temperature and the heating time at each stage can be appropriately set according to the measurement sample.

TABLE 1
Example of Heating Condition in Heating
Temperature Determination Mode

| Temperature Stage | 1 | 2 | 3 | 4 | 5 | 6 |
|---|---|---|---|---|---|---|
| Temperature (°C) | 100 | 120 | 140 | 160 | 180 | 200 |
| Heating Time (min) | 5 | 5 | 5 | 5 | 5 | 5 |

The heating condition in the heating temperature determination mode may be stored in advance in the storage unit 8. The storage unit 8 may be configured to store a plurality of sets of heating conditions according to the types of samples. In this case, users may select a suitable one to set the heating condition. Alternatively, the moisture meter 100 may be configured to allow the user to manually input and set desired conditions on an individual basis.

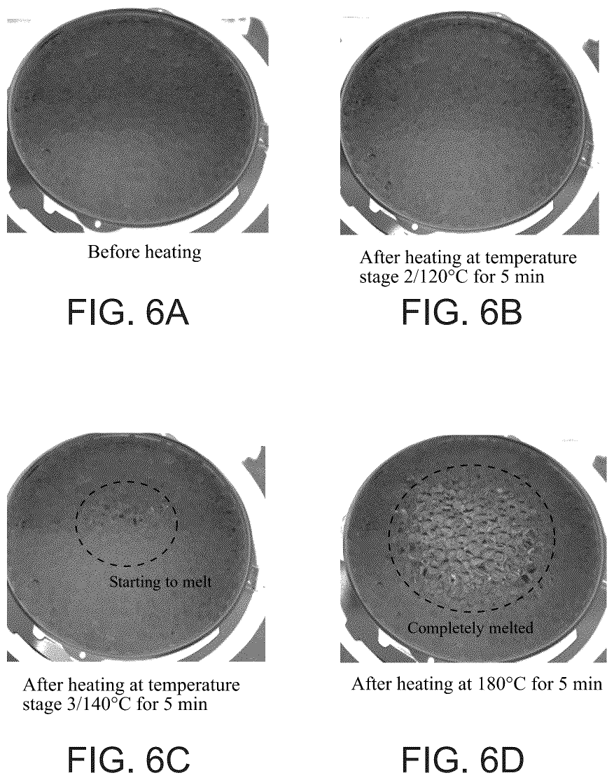

Next, the heating temperature determination unit 52 records the state of the preliminary test sample for each temperature stage. When recording a change in the state (appearance) of the preliminary test sample, the heating temperature determination unit 52 determines the temperature at the immediately preceding stage to a heating temperature to be used in the sample measurement mode. Assume that the sample is resin pellets. Heating such sample beyond the glass transition point or the melting point may causes a change in the appearance. For example, the appearance changes from translucent to transparent, or from transparent to milky white. For example, polypropylene resin pellets exhibit milky white color (see, for example, FIG. 6A described later), but the pellets melt and become transparent as the heating temperature is increased (see, for example, FIG. 6D).

In addition, scorching or discoloring sometimes occurs. In this specification, the term “state change” refers to such a change in appearance. Such state changes cause the evaporation of components other than moisture in the sample, thereby hindering the accurate measurement of moisture content. In the present embodiment, a temperature at a stage immediately preceding a temperature stage at which such a change has occurred is set as a heating temperature. This enables measurements at a temperature that prevents such state changes.

The storage unit 8 is implemented using a non-transitory computer-readable storage medium such as a hard disc drive (HDD), a solid state drive (SDD), or a flash memory. The storage unit 8 stores various programs for the arithmetic control unit 5 to execute the functions of the functional units.

The timer 12 measures the time from the start of heating and outputs the end signal to the arithmetic control unit 5 when the predetermined time set by the arithmetic control unit 5 has elapsed.

### (Heating Temperature Determination Mode)

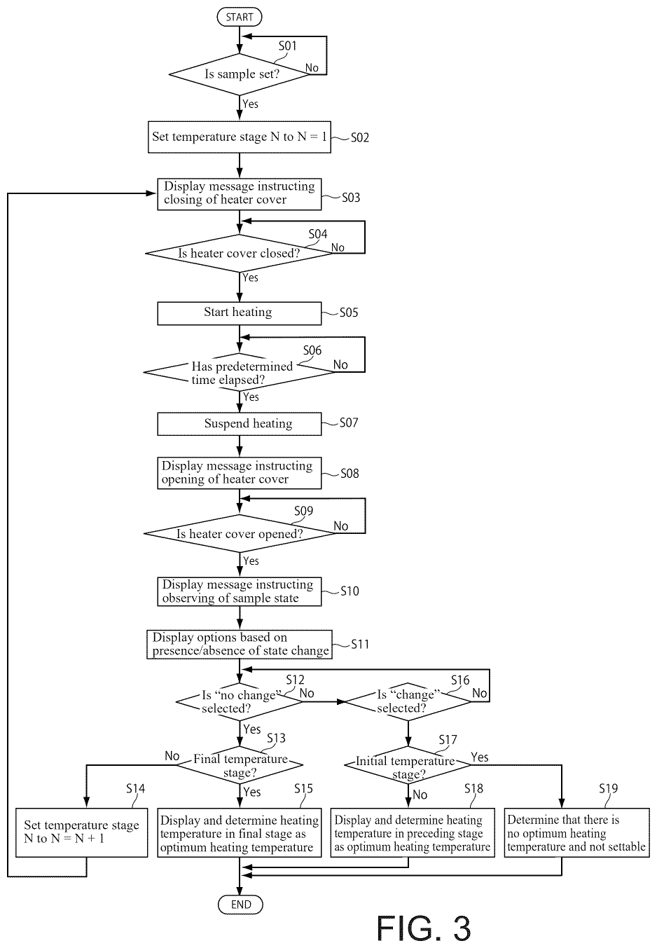

Next, the heating temperature determination mode will be described. FIG. 3 is a flowchart of an example of the operation of the moisture meter 100 in the heating temperature determination mode. The heating temperature determination mode is a mode for determining the heating temperature in the sample measurement mode, which is executed prior to the execution of the sample measurement mode. The heating temperature determination mode starts when the user selects the heating temperature determination mode from the mode selection screen displayed on the display unit 7, for example. During the execution of the heating temperature determination mode, the heating temperature determination unit 52 constantly calculates the mass based on the detection signal from the mass sensor 2.

When starting the processing, in step S01, the heating temperature determination unit 52 determines whether a sample has been set. When the user sets a preliminary test sample placed on the sample pan 2a in the pan receiver, the heating temperature determination unit 52 determines that the sample is set (“Yes” branch in step 01.) based on a detection signal from the mass sensor 2 and sets the temperature stage N to N=1 in step S02.

Next, in step S03, the heating temperature determination unit 52 displays a message instructing the user to close the heater cover 9a on the display unit 7. In response to this, the user closes the heater cover 9a. When detecting that the heater cover 9a is closed based on a detection signal from the open/close sensor 4 (“Yes” branch in step S04), the heating temperature determination unit 52 proceeds to step S05 to start heating at the temperature at temperature stage 1 in the heating condition. When starting heating, the heating temperature determination unit 52 monitors the moisture content based on a signal from the mass sensor 2 in real time. The heating temperature determination unit 52 also calculates and monitors the change rate of the moisture content in the same manner.

When the set predetermined time has elapsed (“Yes” branch in Step S06), the heating temperature determination unit 52 stops the heating unit 3 to suspend heating in step S07.

Next, in step S08, the heating temperature determination unit 52 causes the display unit 7 to display a message instructing the user to open the heater cover 9a. In response to this, the user opens the heater cover 9a. When detecting that the heater cover 9a has been opened based on a detection signal from the open/close sensor 4 (“Yes” branch in step S09), the heating temperature determination unit 52 proceeds to step S10 to cause the display unit 7 to display a message instructing the user to observe the state of the sample The user observes the state of the sample accordingly.

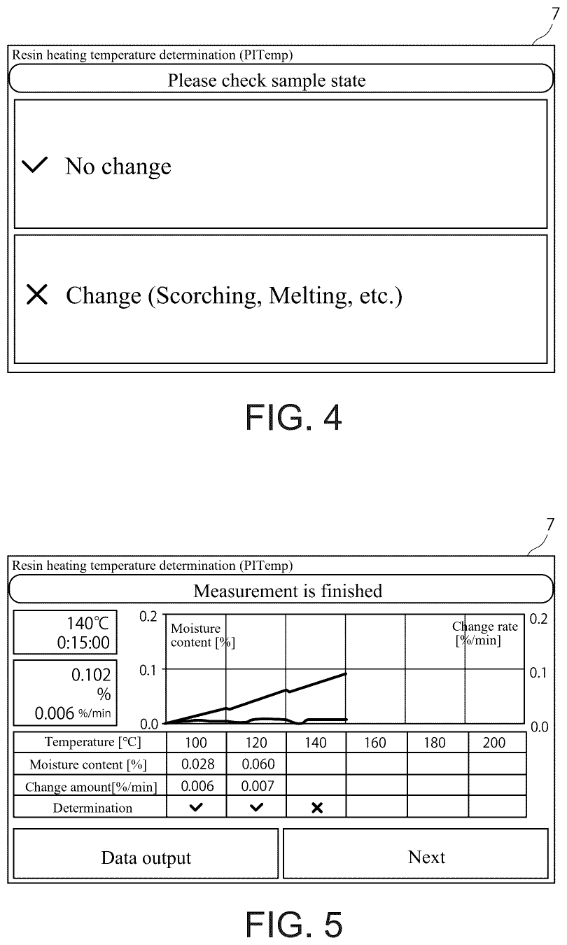

Subsequently, in step S11, the heating temperature determination unit 52 displays options based on whether the preliminary test sample undergoes a state change. FIG. 4 illustrates an example of a screen displaying the message in step S10 and the options based on the presence or absence of state changes displayed on the display unit 7. The user taps to selects an option corresponding to the observed state of the sample.

When “no change” is selected (“Yes” branch in step S12), the heating temperature determination unit 52 proceeds to step S13 and determines whether the current temperature stage is the final temperature stage. When the current temperature stage is not the final (“No” branch in step S13), the heating temperature determination unit 52 proceeds to step S14, sets the temperature stage N to N=N+1, and then returns to step S03. Determining that the current temperature stage is the final (“Yes” branch in step S13), the heating temperature determination unit 52 proceeds to step S15. The heating temperature determination unit 52 determines the heating temperature at the final stage as an appropriate heating temperature, displays it on the display unit 7 and sets it to the heating temperature in the sample measurement mode to be performed next. Then, the heating temperature determination unit 52 terminates the processing.

On the other hand, when “no change” is not selected (“No” branch in step S12), the heating temperature determination unit 52 determines whether “change” is selected in step S16. When “change” is selected (“Yes” branch in step S16), the heating temperature determination unit 52 determines whether the current temperature stage is the first temperature stage, i.e., temperature stage 1, in step S17.

When determining that the current temperature stage is not the first temperature stage, in other words, that the current temperature stage is temperature stage 2 or later (“No” branch in step S17), the heating temperature determination unit 52 proceeds to step S18 to determine the temperature at the immediately preceding stage as the appropriate heating temperature. Then, the heating temperature determination unit 52 displays the determined appropriate heating temperature on the display unit 7 and sets it to the heating temperature in the sample measurement mode to be performed next and then terminates the processing.

On the other hand, when the current temperature stage is the first temperature stage (temperature stage 1) (“Yes” branch in step S17), the heating temperature determination unit 52 proceeds to step S19t to determine that there is no appropriate heating temperature for measuring a sample which is the same substance as the preliminary test sample, display a message indicating that determination of the heating temperature has failed on the display unit 7, and then, terminate the processing.

FIG. 5 illustrates an example of the screen displayed on the display unit 7 at the end of measurement when the process proceeds to step S18. FIGS. 6A to 6C are photographs showing the state of the sample at each temperature stage in FIG. 5. FIG. 6D shows, as a reference, a state in which the same resin pellets as that in FIGS. 6A to 6C are completely melted by heating at 180 °C for 5 min. Note that the resin pellets is polypropylene.

As illustrated in FIG. 5, the display unit 7 displays, as a result screen, a moisture content-time profile in the form of a graph. The display unit 7 updates the moisture content at the end of each temperature stage, the change rate of the moisture content, and the determination results of the state of the sample in the form of a table. With such a screen, the user can easily grasp the entire measurement results.

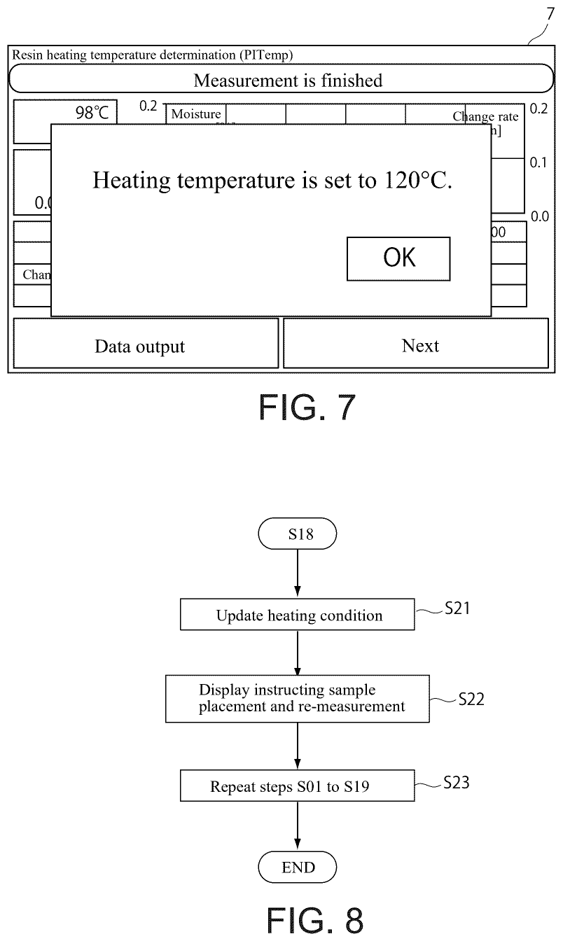

In the result shown in FIG. 5, no state change occurs at temperature stage 1 (100 °C) and temperature stage 2 (120 °C). Actually, as shown in FIG. 6B, no change in the state of the sample occurs between before heating shown in FIG. 6A and the end of temperature stage 2. The heating temperature determination unit 52 determines that a change in the state of the sample has occurred at temperature stage 3 (140 °C). In fact, FIG. 6C shows that the resin pellets partially start to melt and become transparent. Thus, the moisture meter 100 records the determination result of the change in the appearance of the resin pellets by the user at each temperature stage, and terminates the measurement when the change in the appearance is recorded. In this case, the moisture meter 100 determines a temperature of 120 °C at the immediately preceding stage (temperature stage 2) to the temperature stage 3, as the appropriate heating temperature. FIG. 7 illustrates an example of the heating temperature determination screen displayed on the display unit 7.

The moisture meter 100 may be configured such that users can select such heating temperature determined in this manner as an optimal heating temperature at the start of the sample measurement mode. Alternatively, moisture meter 100 may be configured to automatically set the determined heating temperature as a heating temperature to be used in the sample measurement mode. This processing may be executed in steps S15 and S18. Alternatively, this processing may be executed as a part of processing of the sample measurement mode.

Note that the display unit 7 in the present embodiment corresponds to an output interface in the claims, but the output interface is not limited to the display unit 7. For example, the moisture meter 100 may further include an audio output unit that notifies the user of information by a voice message and is configured to notify the messages in steps S03, S08, and S10 by the voice output unit in addition to or instead of the display on the display unit 7. Alternatively, the moisture meter 100 may further include a communication unit and be configured to display these messages not only on the display unit 7 but also on a display unit of an external device, such as a computer or a smartphone, connected via the communication unit.

### Operation and Effect

The moisture meter 100 according to the present embodiment is configured to heat the preliminary test sample while raising the heating temperature stepwise and record the state of the preliminary test sample at each temperature stage. This allows a determination of the temperature at which the state of the sample changes by one measurement. Determining the temperature at the immediately preceding temperature stage immediately preceding a temperature stage at which the change in the state of the preliminary test sample occurs as a heating temperature to be used in the sample measurement mode allows to easily determine an appropriate heating temperature at which the measurement sample does not melt or scorch during measurement. The present moisture meter 100 and method are particularly suitable for use as a sample with low moisture content, such as resin pellets.

Generally, with a conventional moisture meter equipped with a heater cover, it is impossible to observe the state of the sample when the heater cover is closed. Furthermore, such a type of moisture meter is generally configured to terminate a measurement with error if the heater cover is opened during the measurement. On the other hand, the moisture meter 100 according to the present embodiment is configured to allow users to open the heater cover and observe the state of the sample between the temperature stages and to guide users to observe the state of the sample via the output interface. This allows the moisture meter 100 to determine the appropriate heating temperature by receiving the determination result of the state of the sample, from the user according to the instructions by the moisture meter 100.

### First Modification

The moisture meter 100 according to the present embodiment may be modified as follows. FIG. 8 is a flowchart illustrating one modification of the process in the heating temperature determination mode of the moisture meter 100.

This modification includes a determination more detailed heating temperature after determining the heating temperature as described above in the moisture meter 100 according to the first embodiment. In this modification, the moisture meter 100 executes an additional step illustrated in FIG. 8 after step S18 illustrated in FIG. 3.

Specifically, after determining the temperature at the immediately preceding temperature stage as the heating temperature in step S18, in step S21, the heating temperature determination unit 52 updates a heating condition by further subdividing the temperature interval between the temperature of the immediately preceding temperature stage and the temperature of the temperature stage where the state change of the sample has occurred.

For example, as in the above example, when the state change has occurred at the temperature stage 3 (140 °C) and the immediately preceding temperature stage is the temperature stage 2 (120 °C), the heating temperature determination unit 52 updates the heating condition to have intervals of 5 °C between 120 °C and 140 °C. That is, in the updated heating condition, temperature stage 1 is set to 120 °C, temperature stage 2 is set to 125 °C, temperature stage 3 is set to 130 °C, temperature stage 4 is set to 135 °C, and temperature stage 5 is set to 140 °C.

Next, in step, the heating temperature determination unit 52 displays a message instructing the user to remove the measured preliminary test sample, place a new preliminary test sample, and perform re-measurement on the display unit 7. Then, in step S23, the heating temperature determination unit 52 repeats steps S01 to S19 under the updated heating condition, and then, terminates the processing.

In this way, the obtained heating temperature is a higher temperature at which the measurement sample can be heated without melting or discoloring. In the present modification, the appropriate heating temperature can be determined more accurately.

### Second Modification

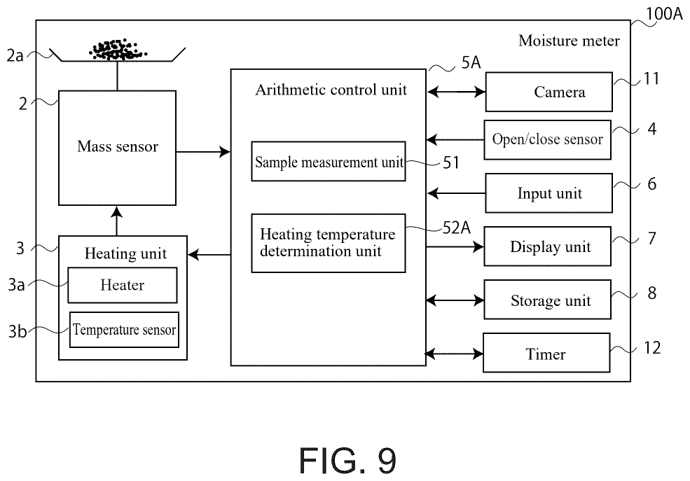

FIG. 9 is a configuration block diagram of a moisture meter 100A according to another modification of the moisture meter 100. The moisture meter 100A generally has the same configuration as the moisture meter 100. However, the moisture meter 100A differs from the moisture meter 100 in that it further comprises a camera 11, and comprises an arithmetic control unit 5A instead of the arithmetic control unit 5, the arithmetic control unit 5A having a mechanical configuration equivalent to that of the arithmetic control unit 5 but having a heating temperature determination unit 52A instead of the heating temperature determination unit 52.

The camera 11 comprises an imaging element such as a charge-coupled device (CCD) or a complementary metal-oxide-semiconductor (CMOS); a lens; and an illumination such as a light emitting diode (LED). The camera 11 is provided inside the cover 9 and acquires images of the sample placed on the sample pan 2a in the closed state of the heater cover 9a which is provided inside the cover 9, to output it to the arithmetic control unit 5A.

The heating temperature determination unit 52A executes the heating temperature determination mode similarly to the heating temperature determination unit 52. However, the heating temperature determination unit 52 records the change in state of the sample by instructing the user to observe the sample whereas the heating temperature determination unit 52A evaluates the change in state based on the images acquired by the camera 11 and, when the change has occurred, records the states of the sample and determines a temperature at the immediately preceding temperature stage as the heating temperature to be used in the sample measurement mode.

The heating temperature determination unit 52A can determine the change in state of a preliminary test sample by using a learning model for determining the change in state of resin pellets, for example, the learning model being generated by learning a large number of images of resin pellets before and after heating that has caused state changes, using a convolutional neural network (CNN). The learning model may be generated by using a support vector machine, Random Forest, the K-nearest neighbor algorithm, etc., as well as CNN. In addition, the heating temperature determination unit 52A may be configured by a known image determination technique that does not use any learning model.

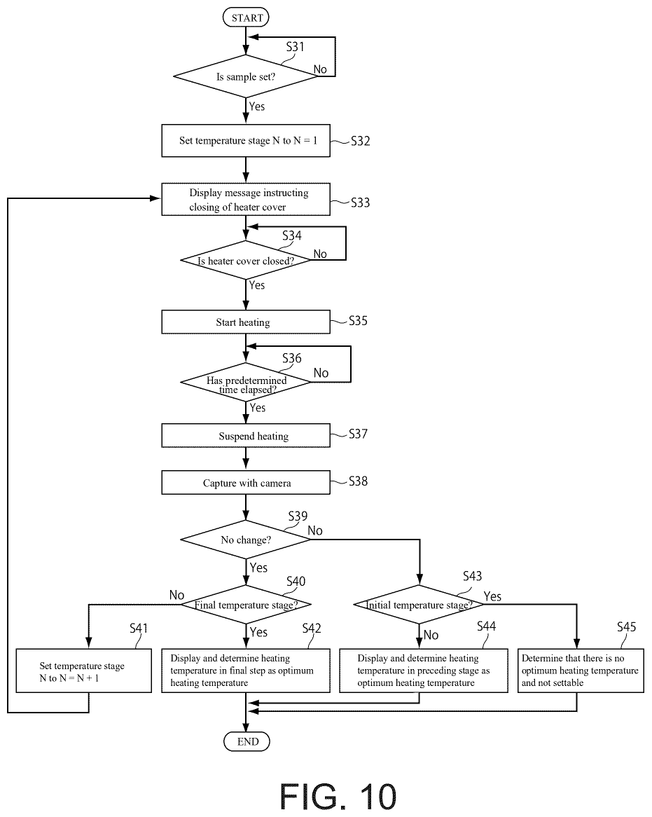

FIG. 10 is a flowchart illustrating an example of processing by the heating temperature determination unit 52A in the heating temperature determination mode. Since steps S31 to S37 are the same as steps S01 to 07, a description thereof is omitted. After step S37, in step S38, the heating temperature determination unit 52A drives the camera 11 to capture the state of the sample. Next, in step S39, the heating temperature determination unit 52A determines whether there is a change in the state of the sample. Upon determining that there is no change, the heating temperature determination unit 52A determines in step S40 whether the current temperature stage is the final temperature stage and then executes the same processing as that in steps S14 and S15 in steps S41 and S42, respectively. On the other hand, upon determining that there is a change in step S39, the heating temperature determination unit 52A determines in step S43 whether the current temperature stage is the first temperature stage, and then executes the same processing as in steps S18 and S19 in steps S44 and S45. Such a configuration allows automatic determination of the appropriate heating temperature without making users open the heater cover 9a for each temperature stage and observe the sample. In addition, since the moisture meter 100A does not need for suspending heating during execution of the heating temperature determination mode, thereby shortening the time required for determining a heating temperature.

Although the moisture meter 100A is configured to examine the appearance of the preliminary test sample at the end of each temperature stage, the disclosure is not limited to this. The moisture meter 100A may be modified to capture an image at predetermined intervals during heating to examine changes in appearance and determine, when evaluating that a change has occurred, the temperature at the temperature stage immediately preceding the change as an appropriate heating temperature used in the sample measurement mode.

### 2. Second Embodiment

Incidentally, standard methods for measuring moisture content include the dry weight method and the Karl Fischer method. These are considered to have higher measurement accuracy than the methods using moisture meters. For the same sample, the values obtained by moisture meters tend to be higher than those obtained by the standard methods. Therefore, many users desire moisture meters to output measurement values close to the measurement values obtained by standard methods as target values.

Some commercially available moisture meters are configured to multiply a measurement result by a coefficient to output the resultant measurement value as a value close to a target value. However, such a method has low reliability and validity. Alternatively, measurement conditions are variously adjusted to output a measurement value as a value close to a target value, but a large number of measurements are required to determine the measurement conditions, resulting in complicated work.

As a result of intensive studies, the inventors have found that when a sample is heated at an appropriate heating temperature for a long time using a moisture meter, a moisture content-time profile shows a constant curve, and by setting a timer end time corresponding to a target value, the resultant value can be output as a moisture content which is a target value with good reproducibility.

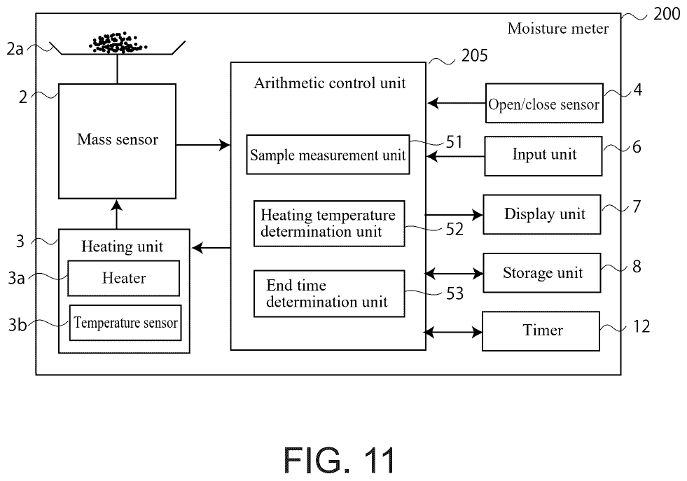

FIG. 11 is a configuration block diagram of a moisture meter 200 according to the second embodiment. The moisture meter 200 schematically has the same configuration as the moisture meter 100 but includes an arithmetic control unit 205 instead of the arithmetic control unit 5. The arithmetic control unit 205 has the same hardware configuration as the arithmetic control unit 5 but differs therefrom in that it includes a heating end time determination unit (hereinafter referred to as “end time determination unit”) 53 in addition to the functional units of the arithmetic control unit 5.

The end time determination unit 53 executes an end time determination mode for determining a heating end time for outputting moisture content at a target value. The end time determination unit 53 sets the target value of moisture content based on a user's input, and heat the sample at a set heating temperature for the predetermined time (for example, 30 min) set by the timer. Starting heating, the end time determination unit 53 sequentially calculates the mass of the sample and calculates the moisture content based on the difference between the mass during heating and the mass before heating, to monitor the moisture content. The end time determination unit 53 stops heating after a predetermined time, calculates the time by which the moisture content set from the moisture content-time profile becomes the target value and determines the calculated time as a measurement heating time. This time is set to a heating time to be used in the sample measurement mode.

### (End Time Determination Mode)

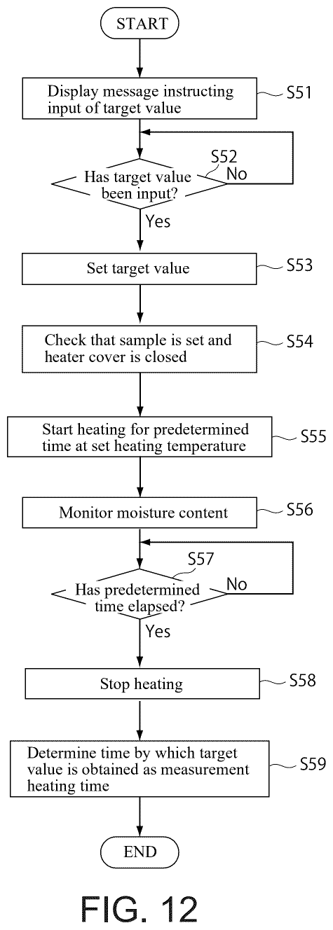

FIG. 12 is a flowchart illustrating an example of the operation of the moisture meter 200 in the end time determination mode. The end time determination mode is performed after the determination of a heating temperature in the heating temperature determination mode but prior to the execution of the sample measurement mode. The end time determination mode is started, for example, by selecting the end time determination mode by the user through the mode selection screen displayed on the display unit 7. In the end time determining mode, the appropriate heating temperature determined in the heating temperature determining mode is preset.

When starting the processing, in step S51, the end time determination unit 53 displays a message instructing a user to input a target value on the display unit 7. When the target value is input in step S52, the end time determination unit 53 sets a target value in step S53.

Next, when confirming that the sample is set and the heater cover 9a is closed in step S54, in step S55, the end time determination unit 53 starts heating for a predetermined time at the heating temperature determined in heating temperature determination mode by controlling the heating unit 3.

Next, in step S56, the end time determination unit 53 sequentially detects the mass of the sample and calculates the moisture content of the sample based on the difference between the mass during heating and the mass before heating, to monitor the moisture content. When the predetermined time has elapsed in step S57, the end time determination unit 53 stops heating of heating unit 3 in step S58.

Next, in step S59, the end time determination unit 53 calculates the time by which the moisture content reaches the target value from the moisture content-time profile obtained as a result of a series of monitoring and determines the calculated time as the measurement heating time for outputting the moisture content as the target value. Then, the end time determination unit 53 displays the measurement results and terminates the processing. The display screen of the measurement result displays the moisture content-time profile and the determined measurement heating time.

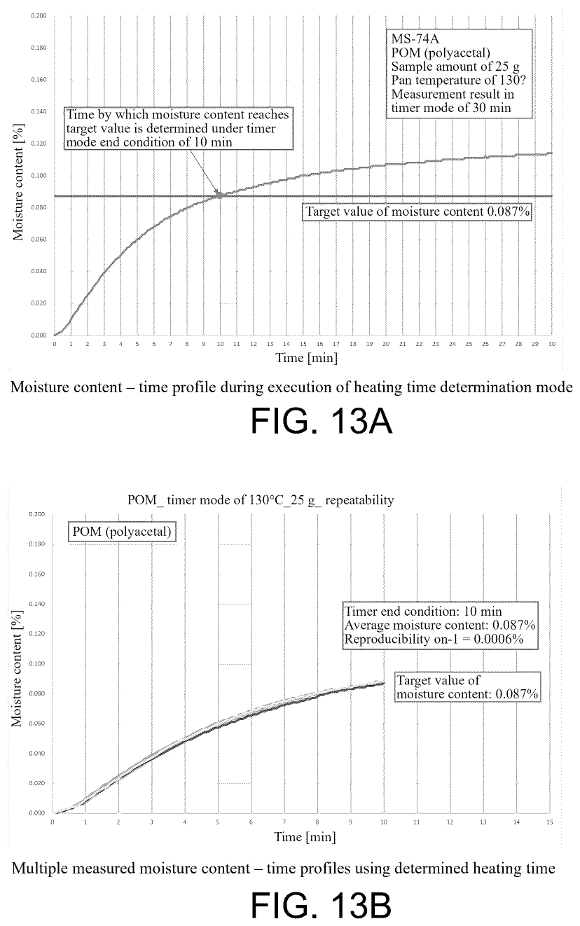

FIG. 13A is, as an example, a moisture content-time profile when resin pellets 25 of polyacetal are heated in a timer mode in which the heating time is set for 30 min. The heating temperature is 130 °C, which is the value determined in the heating temperature determination mode. Here, the moisture content value of the polyacetal resin pellets measured by the Karl Fischer method is 0.087%. Therefore, assuming that the target value of the moisture content is 0.087%, the heating time of 10 min by which the measured moisture content becomes 0.087% is determined as the measurement heating time.

FIG. 13B is a moisture content-time profile illustrating the result of performing the sample measurement mode a plurality of times using the measurement heating time determined using the result in FIG. 13A and the heating temperature determined in the heating temperature determination mode. FIG. 13B illustrates that the measurement moisture content can be output as a target value with good reproducibility by using the measurement heating time determined in this manner and the appropriate heating temperature.

The target value thus set may be stored in a storage unit 8 together with the measurement heating time determined (estimated) by the method disclosed in Patent Literature 2, for example. Setting the determined measurement heating time as a measurement heating time option at the time of executing the sample measurement mode enables the moisture meter 200 to output a measurement value as moisture content matching the target value according to the purpose of measurement. Note that the target value is not limited to the moisture content determined by the standard method described above, and a moisture content value with desired measurement accuracy may be used.

Although the preferred embodiments of the present disclosure have been described above, the embodiments are just examples of the present disclosure, and these can be combined based on knowledge of a person skilled in the art, and such a combined embodiment is also included in the scope of the present disclosure.

In addition, although the present specification has described a heat-drying moisture meter as a moisture meter, the present disclosure can also be applied to a moisture meter of another form that includes a mass sensor and detects a change in the mass of a sample before and after heating to calculate a moisture content.

### REFERENCE SIGNS LIST

2: Mass sensor
2a: Sample pan
3: Heating unit
5, 5A, 205: Arithmetic control unit
6: Input unit
9a: Heater cover
25: Resin pellets
51: Sample measurement unit
52: Heating temperature determination unit
53: End time determination unit
100, 200: Moisture meter
M: Moisture content

1. A moisture meter comprising:
a mass sensor configured to measure a mass of a sample placed on a sample pan;
a heater configured to heat the sample; and
an electronic circuit configured to control the heater to heat the sample at a set temperature and calculate moisture content of the sample from a difference in mass of the sample before and after heating,
wherein the electronic circuit is configured to execute a heating temperature determination mode of:
heating a preliminary test sample for a predetermined time at each temperature stage while raising a heating temperature stepwise;
recording a state of the preliminary test sample at each temperature stage; and
when a change in the state of the preliminary test sample occurs, determining a temperature at an immediately preceding temperature stage as a set heating temperature for a measurement sample, and
wherein the preliminary test sample is the same substance as the measurement sample.

2. The moisture meter according to claim 1, further comprising:
an openable/closable heater cover configured to cover the sample pan to form a heating chamber;
an output interface configured to output information to a user; and
an input interface configured to receive information from a user,
wherein the electronic circuit instructs a user to open the heater cover to observe the state of the preliminary test sample at an end of each temperature stage via the output interface and to input the state via the input interface, to record the state.

3. The moisture meter according to claim 1, wherein the measurement sample is resin pellets.

4. The moisture meter according to claim 1, wherein the electronic circuit is configured to update a heating condition with smaller temperature intervals between a temperature stage at which the change in the state of the preliminary test sample occurs and the immediately preceding temperature stage and to execute the heating temperature determination mode again using an updated heating condition.

5. The moisture meter according to claim 1,
wherein the electronic circuit is configured to:
set a target value of the moisture content of the measurement sample;
heat the preliminary test sample at the set heating temperature for a predetermined time while monitoring the moisture content of the preliminary test sample; and
set a time by which the moisture content reaches the target value as a measurement heating time.

6. The moisture meter according to claim 1, further comprising a camera configured to capture images of the preliminary test sample being measured at predetermined intervals,
wherein the electronic circuit is configured to
examine a state of the preliminary test sample based on the images and
determine the temperature at the immediately preceding temperature stage immediately preceding to the temperature stage at which the change in the state of the preliminary test sample occurs as the set heating temperature for the measurement sample.

7. A method for controlling a moisture meter, executed by a computer serving as an arithmetic control unit of the moisture meter that heats a sample at a set temperature and calculates a moisture content of the sample from a difference in mass of the sample before and after the heating, the method comprising:
heating a preliminary test sample for a predetermined time at each temperature stage while raising a heating temperature stepwise;
recording a state of the preliminary test sample at each temperature stage; and
when a change in the state of the preliminary test sample occurs, determining a temperature at an immediately preceding temperature stage as a heating temperature for a measurement sample,
wherein the preliminary test sample is the same substance as the measurement sample.

8. A non-transitory computer-readable storage medium including a program for causing a computer serving as an arithmetic control unit of the moisture meter to execute the method defined in claim 7.
